(19) United States
(12) Patent Application Publication
(10) Pub. No.: US 2025/0134692 A1
(43) Pub. Date: May 1, 2025

(54) Self contained, Portable Female External Urine Catheter

(71) Applicants: Zachary Walter Newman, Van Nuys, CA (US); Ellie Shana Harooni, Encino, CA (US)

(72) Inventors: Zachary Walter Newman, Van Nuys, CA (US); Ellie Shana Harooni, Encino, CA (US)

(21) Appl. No.: 18/445,573

(22) Filed: Oct. 25, 2023

(51) Int. Cl. A61F 5/44; A61F 5/441; A61M 25/00; A61M 25/02

(52) U.S. Cl. CPC A61F 5/4408; A61F 5/4401; A61F 5/4407; A61F 5/441; A61M 25/0017; A61M 2025/026

(57) ABSTRACT

A new external female urinary catheter is disclosed. This system consists of disposable absorbent pads enclosed in reusable polymer pouch. This system is capable of collecting and transporting urine from a female human for a period of 3 to 12 hours, using capillary action of absorbent material. An opening over the vagina will allow the flow of urine into the collection chamber. This system can be used in standing, sitting, or lying positions. This system can control and neutralize the odor associated with the urine and also sanitize the urine. This system can also contain the leaking urine from an indwelling foley type catheter. This system can alert the nurse to potential urinary problems as well as measure urinary output accurately.

### FIELD OF INVENTION

The present disclosure relates generally to systems, apparatus, and methods for collecting and transporting urine away from the body of a female human.

### BACKGROUND OF INVENTION

The embodiments described herein relate generally to collecting and transporting urine away from the body of a human female. In various circumstances, a woman may have limited or impaired mobility such that typical urination processes are challenging or impossible. For example, a person may experience or have a disability that impairs mobility. A person may have restricted travel conditions such as those experienced by pilots, drivers, and workers in hazardous areas. Additionally, urine collection is needed for monitoring purposes or clinical testing.

Most External urinary Catheters for women are designed to only collect urine in a standing position. Some come in as urine collection devices with countless reincarnations of a form-fitting receptacle over the female genital region, hooked up to a tube and subsequently to a collection receptacle in the form of a urinary bag. The obvious disadvantage of these deigns is that they can only be used in standing position and fail at their function during sitting, sleep time and for use with bed bound patients.

Some other systems come in the form of a disposable/reusable diaper with very low capacity for urine absorption. The normal human urinary output is 80 ml/hr and these designs have failed to take this statistic into account. These designs fail at their function for use periods longer than a few hours. They also fail either to keep the urine away from the skin of the patients or control the odor of the urine.

Many of the better functioning systems that are available rely on vacuum pumps to pump away the urine; however these designs fail at their function when there is no vacuum pump available. Vacuum pumps are readily available in hospital settings yet scantly available in nursing homes residences and automobiles. The portable vacuum pumps that can be used outside of the hospital setting are bulky to wear with heavy electronics prone to mechanical failure and power disruption due to their reliance on batteries. The biggest disadvantage of these systems besides their bulk and weight is that these systems are too expensive to be accessible to average humans. It is not clear if these designs are capable of either containing the odor of urine or if they can they sanitize the urine.

There is a need for a new portable, self contained system that, does not rely on electrical and mechanical power, can be used in standing, sitting and lying position, can contain the odor of the urine, is capable of sanitizing the urine, and is capable of absorbing urine for a full 8 to 12 hours during nighttime sleep, leaving the patient feeling dry all the while keeping the urine away from contact with the Vagina and skin. The improvements herein can accomplish all these objectives while no other known system can.

It is well documented in the medical literature that prolonged urine-skin contact can lead to skin breakdown. This system has beneficial effects in that regard. Furthermore a system that is accessible to poor people as well as the rich, also possesses a substantial benefit. The improvements herein have a solution.

This system also comes with a very unique feature. It has a portal to accommodate a foley type indwelling catheter. Foley indwelling catheters, with long term, can start to leak heavily. This system is capable of intercepting the leaking urine from the Foley type Catheter and keep the patients clean and dry. The foley can also be inserted directly into the collection chamber eliminating the need for a leg or bed urine collection bag. This feature has the potential ability to reduce urinary tract infections as well as increase patient mobility. Thousands of patients suffer from leaking foley type catheters. The improvements herein have a solution.

There are hundreds of thousands of females with spinal cord injuries, many paraplegics, and quadriplegics. Most paraplegics and quadriplegics routinely rely on a care taker for diaper changes and catheter placement and emptying. Many will wait hours in urine soiled diapers and clothes before the care taker arrives to help. The improvements herein will put an end to that misery by keeping the person dry and odorless up to 12 hours need be.

This system can also double up as diaper for nighttime use. The diaper like insert is provided to capture feces during the night for bedbound patients. No known urinary catheter has this versatility; hence this is a novel improvement. Also Hundreds of thousands of female bedbound patients reside at nursing homes. This product will have multiple benefits for them as we will discuss later. If this device were to receive wide adoption by healthcare facilities, which it should because it will ultimately save them money on disposable diapers, labor cost associated with Unlicensed personnel performing diaper changes and wound treatment nurses, it will greatly enhance the life quality of hundreds of thousands of patients across the country. The disclosed system can achieve all these objectives.

### SUMMARY OF INVENTION

A system is disclosed that is suitable for collecting and transporting urine away from the body of a human female that is portable, reusable, and self-contained. The disclosed system includes an assembly that will include absorbent material enclosed in a continuous gusseted pouch made of hydrophobic polymer material, starting at the anterior lower waist, stretching through the perineum and terminating at the lower posterior lower torso at the hipline. The pouch can come in 3 different sizes in order to accommodate different size women. The body facing region of the Pouch located at the genital area is fitted with a form-fitting soft polymer cup shaped to encircle the female genital anatomy. The patient can freely urinate into the pouch via the external form-fitting cup while the absorbent material soaks up the discharge through capillary action. A silicone mesh inserted and fused within the receptacle will keep the absorbent material away from the skin. The receptacle that form fits around the genitals of the female is designed to fit into a hole inside of a girdle type panty enrobing the female waist area. The spandex girdle type garment material will be fitted with two buttons in front and one at the back in order to secure the urine discharge receptacle and hold it in place securely. Furthermore the spandex girdle type panty will be fitted with pockets in front and back to receive the top of the pouches, in order to hold them securely in place, creating a semi clandestine appearance. The interior, patient facing part of the apparatus can be protected from feces discharge at night by use of a diaper like insert. The two ends of the pouch are fitted with a zippers or flaps that control the unpleasant odor of the urine. The zippered tops also allows for changing of pads; hence transporting the urine away from the body. The edges of the pads may be impregnated with a mild body safe deodorant that will further reduce the odor of the urine. The edges of the pads may be doped with crystalline forms of chlorohexadine, sodium hypochlorite, or other body safe antiseptic. The Anterior face of the pouch may be fitted with a portal to accommodate a foley indwelling catheter and intercept the leaking urine. The anterior face of the pouch may have a chart converting weight of the pouch after the fluid collection to milliliters of urinary output. The interior of the pouch may be fitted with a urine diagnostic strip.

### BRIEF DESCRIPTION OF THE DRAWINGS

FIG. 1 Posterior (body facing) view of the hydrophobic collection pouch with the urine receptacle attached

FIG. 2 Anterior view of the pouch depicting the way Pads are inserted

FIG. 3 Posterior perspective view of the pouch with the meatus receptacle fused

FIG. 4 The meatus receptacle, with the mesh inserted

FIG. 5 The Anterior view of the whole apparatus, The combination of receptacle and the Girdle type holster

FIG. 6 The posterior view of the whole apparatus, the combination of receptacle, pouch and Girdle type holster

FIG. 7 The side view of the whole apparatus as worn in standing position

FIG. 8 The side view of the apparatus as worn in supine position

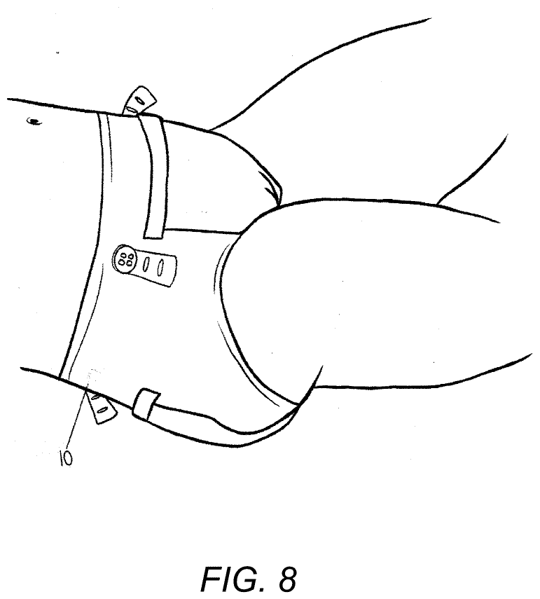

FIG. 9 The anterior view of the apparatus with a diaper like insert

FIG. 10 The side view of the apparatus a worn while using a foley catheter

### DETAILED DESCRIPTION

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

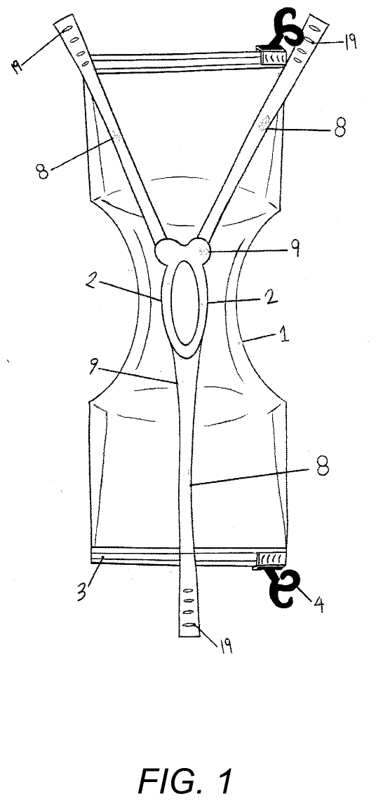

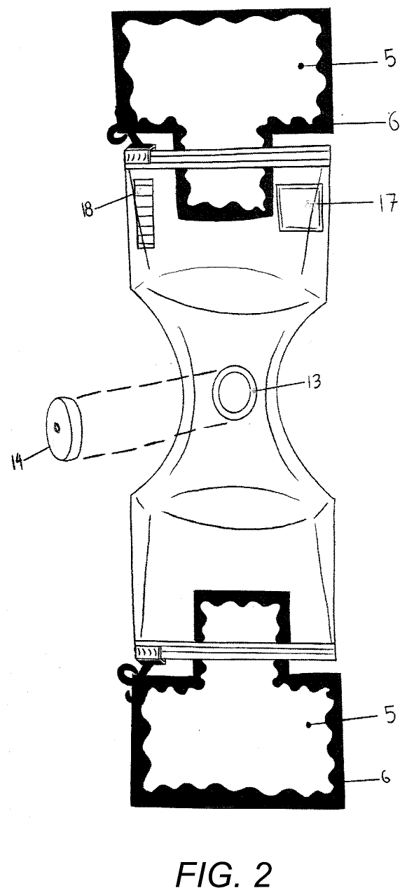

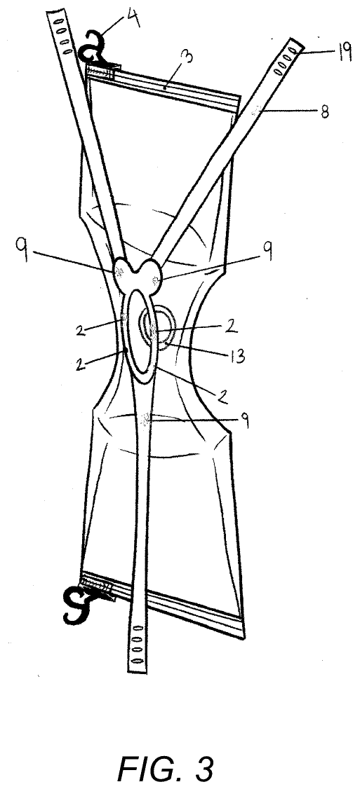

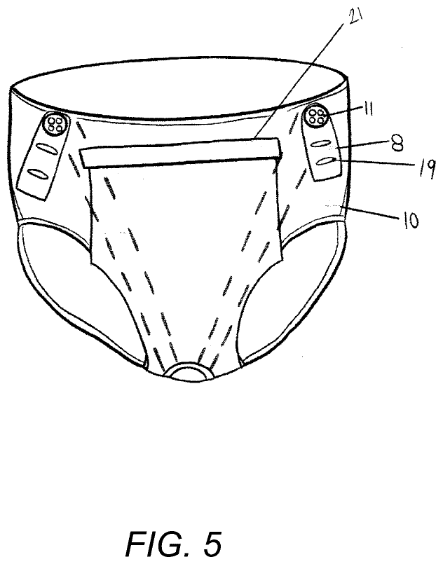

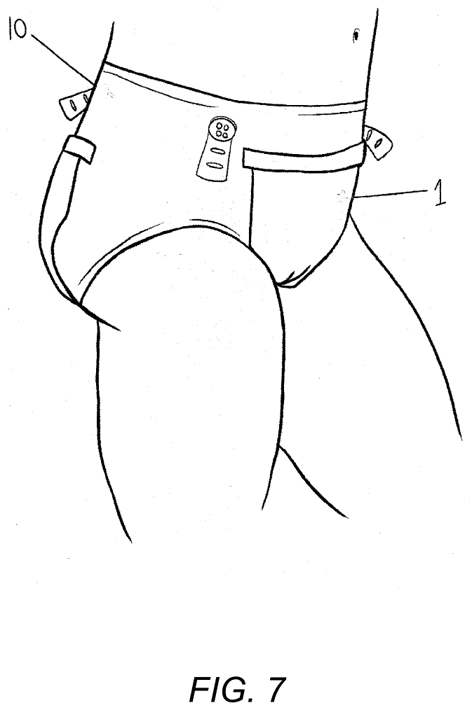

A system is disclosed that is suitable for collecting and transporting urine away from the body of a human female that is portable, reusable, and self-contained. The disclosed system includes an assembly that will include absorbent material 5 see FIG. 2 for the transportation of urine enclosed in a gusseted pouch 1 see FIGS. 1,2,3,5,6,7,8,9,10 made of hydrophobic polymer material for the collection of urine, held in place via a girdle type panty 10 see FIGS. 5,6,7,8,9,10. The continuous pouch 1 wraps around the meatus region of a female starting from the abdomen at the waistline, through the perineum, all the way to the lower torso area terminating at the rear hipline see FIG. 7. This continuous pouch 1 can come in 3 different sizes in order to accommodate children as well as different size adults. In one implementation, the pouch 1 may be 20 cubic inches in size. This system is suitable to collect urine for 5 hours for patients that are only suffering from mild dripping type of urine discharge. On a second implementation, the pouch 1 may be 35 cubic inches in size. This implementation is suitable to collect urine for up 9 hours on a low discharge level and up to 4 hours for a normal Urinary output level of 80 milliliters per hour. On a third implementation, the pouch 1 may be sized at 70 cubic inches. This system is designed to replace an adult diaper and can collect up to 12 hours of urine at normal human urinary output level of 80 ml/hr. All three implementations are suitable for 24 hour use, awake or sleep, ambulatory or non-ambulatory states. The pouch 1 is fitted with a zippers or flap type closures 3 on both anterior and posterior regions in order to control and reduce the dispersion of the urine odor into the patients' surroundings see FIG. 3. The zipper 3 tops also allows for changing of pads; hence transporting the urine away from the body. The Handle of the zipper 3 may have a large bidirectional S shaped handle 4 suitable for paraplegics and quadriplegics with limited use of their upper digits see FIGS. 1,2,3. Examples of polymers include nylons, polyesters, polycarbonates, polyacrylates, polymers of ethylene-vinyl acetates and other acyl substituted cellulose acetates, non degradable polyurethanes, polystyrenes, polyvinyl chloride, polyvinyl fluoride, poly (vinyl imidazole), chlorosulphonate polyolefins, polyethylene oxide, polyethylene terephthalate (PET), polytetrafluoroethylene (PTFE), and/or blends and copolymers thereof.

The interior pad 5, the urine transportation device, see FIG. 2, is constructed of absorbent material, woven or non-woven, shaped to fit inside the pouch. The edges 6 of the pad 5 may be impregnated by a body safe deodorant material to neutralize the odor associated with Urine see FIG. 2. The deodorant impregnated material of the pad does not come in contact with the female member until a urination event. This is an extra safety precaution. In some embodiments, the edges 6 of the pads 5 are doped with body safe antiseptic materials to disinfect the urine of patient's with a history of urinary tract infections see FIG. 2. This transportation device is novel and different from a regular absorbent pad. The pads 5 are only doped with antiseptic crystals at the pad edges 6 in order to prevent contact of the antiseptic with the female skin see FIG. 2. Only after a urination event will the crystals dissolve and sanitize the discharged urine. The amount of antiseptic crystals will be adjusted in such way as to create the right aqueous concentration of the antiseptic in order to be body safe. Such disinfectants may be chlorohexadine crystals or sodium Hypochlorite microencapsulation. Safer and more user friendly antiseptics can always be substituted.

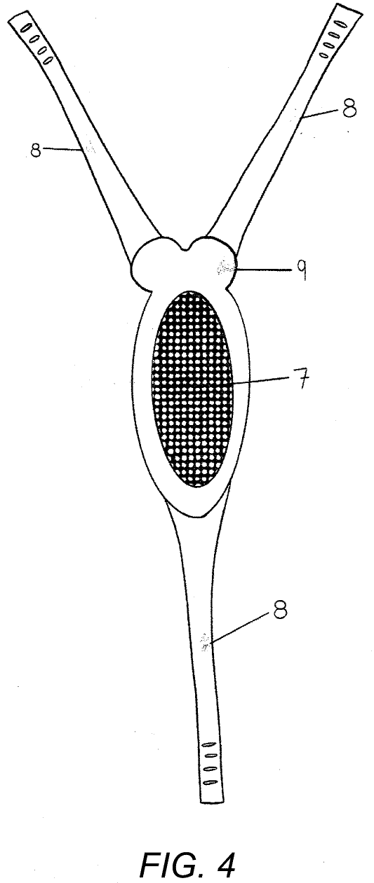

The center of the body facing area of the continuous pouch at the meatus is fitted with a receptacle 2 that will fit tightly against the female genital in order to receive the discharged urine see FIGS. 1,3,4. This solid piece receptacle 2 may be molded in one solid piece and contains 3 flaps 9 and straps 8 see FIG. 4. The function of the straps will be discussed later as part of the system of holding the catheter in place. A silicone mesh 7 will form a barrier between the female skin area and the absorbent material to further enhance the feeling of dryness as well as to prevent prolonged urine-vagina contact see FIG. 4. This Mesh can also be made from materials with antimicrobial properties such as Chlorohexedine doped silicone for patients with a history or urinary tract infections. The receptacle is fused into the pouch 1 with heat sealing methods. The receptacle protrudes ½ inch outside of the pouch in order to be inserted into a special spandex girdle type panty apparatus holder 10 see FIGS. 5,6,7,8,9,10. The receptacle may be made of Silicone or other body safe polymers.

A system has been designed to keep the catheter, which is the combination of the pouch 1 receptacle 2 and pads 5, in place at all times, in all positions, functioning properly. The urine receptacle 2 that fits over the female genitals is fitted with two flaps 9 and three straps 8 see FIG. 4. The straps are made of high strength silicone or rubber or further backed by strong fabric. The straps will have slit type openings 17 in order to tie into a button 11 see FIGS. 5,6,7,8,9,10 onto the spandex Girdle type Holder 10 see FIGS. 5,6,7,8,910. There will be multiple Button tie points on the straps in order to adjust the tightness of the fit over the genital area in order to prevent moving and leaking. The urine receptacle can be detached from its spandex girdle type holder in order to allow for washing of the holder as well as sanitizing the catheter.

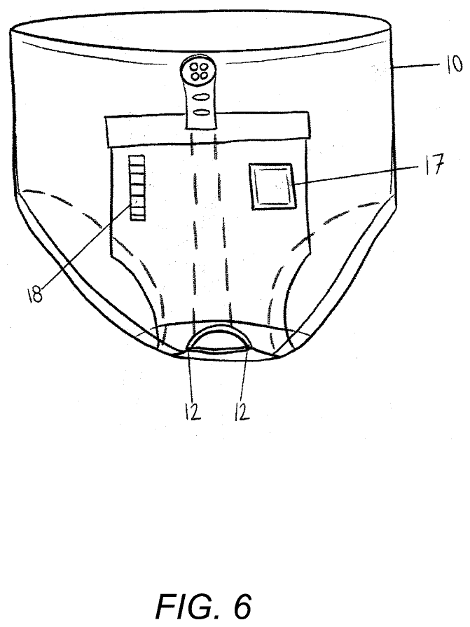

The Catheter, the combination of 1, 2 and 5, can be detached from the holder though a special design. A Hole is created in place where the holder would cover the genitalia of the female. This hole 12 is capable of receiving the catheter from the outside see FIG. 6, around the area covering the female genitals. Once the Catheter is inserted, it is held in place loosely with the butterfly flaps 9 until the final in place securing is made by tying the straps onto the Girdle type holder 10 see FIG. 5. The girdle type holder has pockets 19 in front and back to receive the edges of the catheter see FIG. 5. This feature keeps the catheter use clandestine by disallowing the distal protrusion of pouch 1 edges. Some embodiments of the apparatus, suitable for Healthcare facilities may not have the pockets sewn in on the girdle.

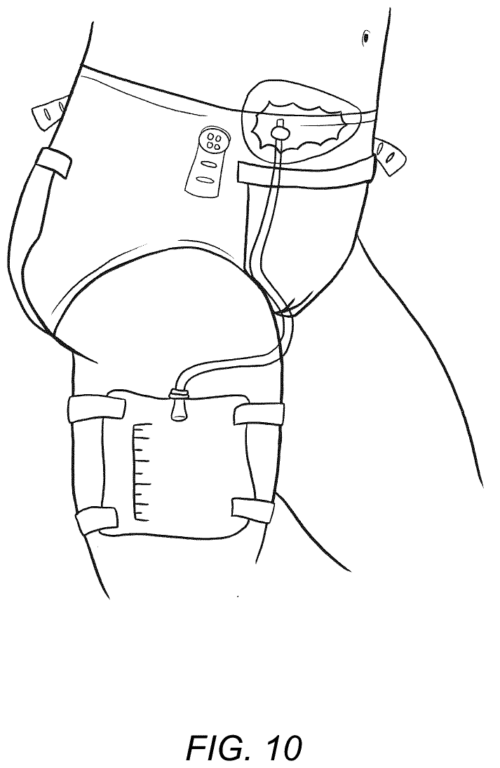

The anterior facing (away from the patient) side of the catheter, in one embodiment, is fitted with a portal 13 see FIG. 2 to receive an indwelling Foley type catheter. Our elegant design is the first of its kind to incorporate such feature. This Embodiment is specifically designed for patients who have suffered from urinary retention for many years and have been using foley type catheters for years. Foley type catheters, with long term use, traumatize the female urethra. This Trauma can prevent a reinsertion of a new foley catheter. Foley catheters often get clogged with sediments inside the urine and need to be flushed and/or replaced. If a new Foley cannot be reinserted the patient remains in a continuously soiled condition. Under this particular situation, our apparatus is an absolute necessity. No other known system can be used in this situation. On other occasions, the foley catheter simply leaks heavily. Our improvements have solved that problem by creating the portal through the pouch, where the leaking urine can be collected by our apparatus. This is achieved easily by passing the the Foley into the cup receptacle from one end and the Foley out through the anterior portal of the apparatus, the Foley extends out towards its tubing 15 see FIG. 10 and finally to its designated urinary collection bag 16 see FIG. 10. In this configuration, any urine that is not collected by the Foley, will be collected by our apparatus, leaving the patient clean, dry, comfortable, free from skin irritation and pressure sores. The portal 14 can simply be capped close when not in use see FIG. 2. Alternatively, if the physician's orders allow, the foley can be inserted directly into the apparatus, eliminating the need for tubing and urine collection bag. This arrangement can significantly improve the mobility and the quality of life of the patient. No other known external catheter has this feature

Our improvements further incorporate two other medically important features. First, the anterior face of the pouch is furnished with a weight to urinary output conversion chart #17 FIG. 2. This is very important because when the catheter is being used as an alternative to diapers in a healthcare setting, the urinary output of the patient can be measured very accurately. As of this writing, healthcare facilities measure the amount of urinary output using the number of diaper changes per shift. This method is grossly inaccurate. With our design, the pouch can simply be weighed on a scale and the conversion table on the pouch will alert the nurse/caretaker to the exact amount of urinary output. The diagnostic capabilities of the apparatus is further enhanced by the incorporation of a urinary diagnostic strip inside the pouch #18 FIG. 2. This strip has the capability to alert the nurse to conditions such as proteinuria, dehydration, and possible urinary tract infections. No other urine collection system offers this flexibility of function.

The most important design feature of our apparatus is the fact that it can be used as a diaper at home, on the road, as well as healthcare setting due to its portability and Self-containment. As to why anyone might want to use our apparatus instead of a diaper, the answer is quite easy. Our apparatus keeps the patient feeling dry as well as neutralizing the odor associated with urine. The catheter is suitable for bedbound patients as well as Paraplegics, and quadriplegics because it uses capillary action of the absorbent material to collect the urine while the patient is at fowlers, supine, prone, or sims positions. No other catheter known without a vacuum pump can make this claim. Even Vacuum assisted catheters restrict the movement of the bedbound patients that is when they are available and affordable. This feature is important for two reasons. First it allows for the timely repositioning of the bedbound patient during day or night. Most doctors' orders require the repositioning of the bedbound Patients every 2 hours in order to prevent bedsores. Secondly, Nursing homes are often understaffed and patients have to wait for hours for a diaper change. To make the matters worse, vacuum assisted collection devices are useless at nursing homes due to lack of vacuum pumps that normally exist at the hospital settings. Meanwhile the skin of the patient is in contact with the wet diaper, slowly being dissolved. Our apparatus keeps the urine away from the surface of the skin for up to 12 hours need be. This feature will greatly reduce the number of skin breakdowns associated prolonged urine-skin contact. It also has the added benefit of freeing the hands of personnel performing diaper changes, in order to perform less diaper changes and more patient repositioning. This arrangement ultimately will greatly reduce the number of bedsores in facilities and the subsequent wound care needs associated with it. Bed sore Wounds are anywhere from unsightly and malodorous to downright painful at the beginning of their formation, not to mention that their treatment costs taxpayers multi-millions of dollars per year. Every healthcare facility has 2-4 nurses specifically dedicated to treating pressure sore wounds.

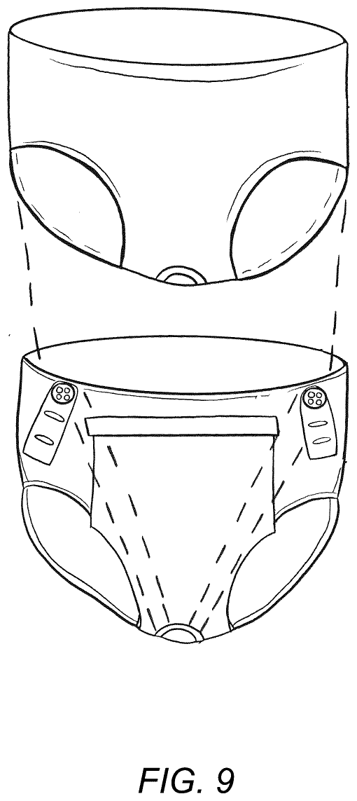

For those patients destined to use this product in a healthcare setting such a nursing home, a nighttime insert 20 will be available see FIG. 9. The insert will capture fecal matter discharged at night. The main function of the insert will be to keep a hygienic environment for the catheter by making clean up easier. The washing of the holder and the catheter will be much easier with this insert in place. Of course, defecation events are far less common at night than urination events. This insert will still allow enough time for the nurse assistants to complete their patient repositioning until a complete catheter change becomes necessary because of the defecation event.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

The recitation of ranges of values herein is merely intended to serve as a shorthand method of referring individually to each separate value falling within the range. Unless otherwise indicated herein, each individual value is incorporated into the specification as if it were individually recited herein, and ranges include their endpoints.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention. Unless a contrary meaning is explicitly stated, all ranges are inclusive of their endpoints, and open-ended ranges are to be interpreted as bounded on the open end by commercially feasible embodiments.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

What is claimed is:

1. A urine collection device, comprising:
a portable collection chamber; and
a urinary meatus receptacle fitted into first wall of the portable collection chamber; and
a holder that holds the collection chamber and meatus receptacle securely in place

2. The device of claim 1, wherein all the walls of the portable collection chamber comprises a waterproof polymer.

3. The device of claim 1, wherein the absorbent pad disposed within the portable collection chamber as well as the collection chamber itself is at least, optionally, 90% biodegradable, either alone or in combination.

4. The device of claim 2, further comprising an absorbent pad disposed within the portable collection chamber

5. The device of claim 2, further comprising an absorbent pad disposed within the portable collection chamber, wherein one of a deodorant crystal or a deodorant compound is disposed at an edge of the absorbent pad.

5. The device of claim 2, further comprising an absorbent pad disposed within the portable collection chamber, wherein one of an antiseptic crystal or an antiseptic compound is disposed at an edge of the absorbent pad.

6. The device of claim 2, further comprising a one of a semi-airtight zipper or a flap type closure disposed on a second wall of the portable collection chamber.

7. The device of claim 2, wherein the collection chamber further comprises a hole configured to receive a portion of an indwelling foley catheter.

8. The devices of claim 1 wherein a female meatus receptacle is fused into the collection chamber as a urine port.

9. The device of claim 8, wherein an edge of the meatus receptacle is molded in one piece incorporating an elastomeric strap.

10. The device in claim 8 wherein the strap of the receptacle has high strength fabric or polymer backing in order to support a perforation that receives a fastening device such as button, eye, hook, eyelet, grommlete or a snap.

11. The device of claim 8, wherein a perforation is created on the strap in order to receive a fastening device such as a button, eye, hook, eyelet, grommlete or a snap.

16. The device of claim 8, wherein the strap of the receptacle is fitted with Velcro type fastener.

17. The device of claim 8, wherein an edge of the meatus receptacle contains a protruding placeholder flap.

18. The device in claim 8, wherein the body facing side of meatus receptacle is covered with silicone mesh.

19. The device in claim 8, wherein the body facing side of meatus receptacle is covered with a mesh compromised of antimicrobial doped material.

20. The device of claim 8, wherein the receptacle further comprises a hole configured to receive a portion of an indwelling foley catheter.

21. Holder in claim 1 wherein a panty made up of spandex girdle type material, with a hole at the perineum area is utilized to hold the devices in claims 2 and 8 in place.

22. The holder device in claim 21 wherein the holder for receptacle and collection chamber has one fastner that connects to a strap initiating from the meatus receptacle.

22. The holder device in claim 21 wherein the holder for the receptacle is fitted with one set of Velcro, hooks and eyes, snaps, hooks and loop, eyelets or grommlets to serve as fastening device in order to fasten the strap originating from the meautus receptacle.
